(19) United States
(12) Patent Application Publication
(10) Pub. No.: US 2011/0196430 A1
(43) Pub. Date: Aug. 11, 2011

(54) SPINAL FIXATION ASSEMBLY WITH INTERMEDIATE ELEMENT

(76) Inventors: David A. Walsh, Reading, MA (US); David S. Randol, Odessa, FL (US)

(21) Appl. No.: 12/703,392

(22) Filed: Feb. 10, 2010

(51) Int. Cl. A61B 17/86

(52) U.S. Cl. 606/305

(57) ABSTRACT

A novel locking mechanism including a body and intermediate element and a method for locking the relative positions of a rod and a screw. The intermediate element is adapted to receive a rod and a head of a screw and is located between the rod and the head of the screw. The intermediate element has a non-circular shape such that at least one large non-contact area exists when the intermediate element is in contact with the head of the screw and the locking mechanism is fully engaged. The body is adapted to maintain the orientation of the non-circular intermediate element with respect to the body. The body also has at least one structure defining at least one distinct temporary stopping point for the intermediate element above the final locking position.

### FIELD OF THE INVENTION

The present invention relates generally to prostheses for treating spinal pathologies, and more specifically to spinal fixation assemblies including a locking mechanism for holding a screw and a stabilization rod.

### BACKGROUND OF THE INVENTION

Various methods of spinal immobilization have been used in the treatment of spinal instability and displacement. The most common treatment for spinal stabilization is immobilization of the joint by surgical fusion, or arthrodesis. This has been known for almost a century. In many cases, however, pseudoarthrosis occurs, particularly in cases involving fusion across the lumbosacral articulation and when more than two vertebrae are fused together. Early in the century, post operative external immobilization, such as through the use of splints and casts, was the favored method of spinal fixation. As surgical techniques became more sophisticated, various new methods of internal and external fixation were developed.

Internal fixation refers to therapeutic methods of stabilization that are wholly internal to the patient and include commonly known devices such as bone plates, screws, rods and pins. External fixation, in contrast, involves at least some portion of the stabilization device being located external to the patients' body. As surgical technologies and procedures became more advanced and the likelihood of infection decreased, internal fixation eventually became the favored method of immobilization since it is less restrictive on the patient.

Internal fixation of the spine may be used to treat a variety of disorders including kyphosis, spondylolisthesis and rotation, segmental instability, such as disc degeneration and/or fracture caused by disease, trauma, congenital defects and tumor diseases. One of the main challenges associated with internal spinal fixation is securing the screw to the spine without damaging the spinal cord. The pedicles of a vertebra are commonly used for fixation as they generally offer an area that is strong enough to hold the screw in place even when the patient suffers from degenerative instability such as osteoporosis.

Current screws and hardware systems used internally for spinal fixation in modern surgical procedures are generally designed to meet one or more criteria, such as: providing rigidity as is indicated, generally along the long axis of the patient's spine; accommodating a broad variation in the size and shape of the spinal member with which it is used; having the capability of handling the stresses and strains to which the devices will be subjected resulting from movement of the spine; and providing easy surgical access during both implantation and removal of the implant.

The present invention includes a novel screw.

### BRIEF SUMMARY OF THE INVENTION

According to aspect of the invention there is provided an intermediate element for use with a locking mechanism, such as a locking mechanism for spinal fixation. The intermediate element may include a top portion adapted to receive a rod and a bottom portion adapted to receive a generally spherical head of a screw. The bottom portion may include at least two feet and each of the feet has a generally curved contact surface at least part of which is adapted to match the contour of a surface of the head of the screw such that a contact area is defined when the foot is brought into contact with the surface of the head. The intermediate element may also include at least one non-contact area between two of the at least two feet spanning at least about 40 degrees of the circumference of the head of the screw. The non-contact area may also have, for example, a width of from about 55 degrees to about 95 degrees of the circumference of the head of the screw

The intermediate element may have a generally non-circular bottom profile, which may be generally ovular. Also, a generally ovular bottom profile may include a generally circular and a generally non-circular part. The generally circular part of the intermediate element may contact the head of the screw, while the generally non-circular part of the intermediate element does not contact the head of the screw.

The intermediate element may include at least two pairs of feet and at least two non-contact areas between the two pairs of feet, wherein each of the two non-contact areas has a width of at least about 40 degrees of the circumference of the head of the screw. In addition, each of contact areas may have a vertical and a horizontal dimension wherein the length of the contact area in the vertical dimension is at least 20 percent of a width of the contact area in the horizontal direction. The sum of the widths of the contact areas on the head of the screw may be less than about 280 degrees of the circumference of the head of the screw.

Also, at least one of the feet may be located at a position generally opposite a position of another of the feet of the intermediate element, or the feet may be offset from one another such that none of the feet is centered at a position opposite a position of a center of another of the feet of the intermediate element.

In addition, the top portion of the intermediate element may be adapted to receive a rod selected from a group of rods of varying diameters.

According to another aspect of the invention, a locking mechanism may include a body having a bottom region and a side adapted to receive a rod; a screw extending at least partially through a hole in the bottom region of the body; and an intermediate element between the rod and the screw, the intermediate element having a top portion adapted to receive a rod and a bottom portion adapted to receive a head of a screw; wherein the body comprises a pocket adapted to receive the intermediate element and maintain the orientation of the intermediate element with respect to the body.

The intermediate element may have a non-circular bottom profile, and the length may be greater than the width. The length may also be generally parallel to a major axis of the rod. The bottom portion of the intermediate element may include at least two contoured surfaces, wherein at least part of each of the contoured surfaces is adapted to match the contour of the surface of the head of the screw to define a contact area on the surface of the head when in contact with the head. In addition, the bottom portion of the intermediate element may include a non-contact area between two of the at least two contoured surfaces, wherein the non-contact area has a width of at least about 40 degrees of the circumference of the head of the screw. The non-contact area may also have a width of from about 50 degrees to about 100 degrees of the circumference of the head of the screw. The at least two of the contoured surfaces may also be opposite one another with respect to a major axis of the intermediate element.

According to another aspect of the invention, a locking mechanism may include a body having a bottom region and a side adapted to receive a rod; a screw extending at least partially through a hole in the bottom region of the body; and an intermediate element adapted for placement between the rod and the screw; wherein locking is accomplished by urging the rod toward the bottom of the body until the intermediate element reaches a final locking position; and wherein the body comprises at least one structure defining at least one distinct temporary stopping point for the intermediate element above the final locking position.

The body of the locking mechanism may include two distinct temporary stopping points for the intermediate element above the final locking position. The at least one structure may impede the progress of the intermediate element toward the bottom of the body, thereby providing feedback to a user that the intermediate element has reached a distinct temporary stopping point. Also, the at least one structure may include a pocket within the body that is wider than the body immediately below the pocket. The intermediate element may be in direct contact with the screw and the rod when the intermediate element reaches the final locking position. In addition, the intermediate element may be not in contact with the screw when the intermediate element is located at least one stopping point.

According to another aspect of the invention, a method is provided for locking the relative positions of a rod and a screw. The method may include inserting a driver into a body at least partially encompassing a screw and an intermediate element located at or above a distinct temporary stopping point defined by at least one structure in the body above the screw; engaging the driver and the screw via a hole in the intermediate element; driving the screw into a desired location; adjusting the position of the body with respect to the position of the screw; placing a rod in the body and urging the rod toward bottom of the body with sufficient force to cause the intermediate element to pass below the temporary stopping point; and further urging the rod toward the bottom of the body to cause the intermediate element to exert force on the head of the screw, thereby locking the position of the rod with respect to the position of the screw.

The intermediate element may be not in contact with the screw when the screw is engaged by the driver. Also, the intermediate element may be located in a pocket of the body when the screw is engaged by the driver. In addition, the intermediate element may have a non-circular bottom profile and the pocket of the body is adapted to receive the intermediate element and prevent rotation of the intermediate element about a major axis of the body. The intermediate element may be caused to exert force on the head of the screw over at least two contact areas separated by a non-contact area having a width of from about 50 degrees to about 100 degrees of the circumference of the head of the screw. Also, the intermediate element may be caused to exert force on the head of the screw over at least two contact areas that are generally located opposite one another with respect to the head of the screw.

The method may also include urging the intermediate element to a second distinct temporary stopping point defined by at least one structure in the body, the second distinct stopping point being below the first distinct stopping point and above the screw.

According to another aspect of the invention, another method is provided for locking the relative positions of a rod and a screw. The method may include placing a rod in a body at least partially encompassing a screw and an intermediate element located above the screw, wherein the intermediate element has a non-circular bottom profile and the body is adapted to receive the intermediate element and prevent rotation of the intermediate element with respect to the body about a major axis of the body; and urging the rod toward the bottom of the body and causing the intermediate element to exert force on the head of the screw, thereby locking the position of the rod with respect to the position of the screw.

The intermediate element may be caused to exert force on the head of the screw over at least two contact areas separated by a non-contact area having a width of from about 50 degrees to about 100 degrees of the circumference of the head of the screw. Also, the intermediate element may be caused to exert force on the head of the screw over at least two contact areas that are generally located opposite one another with respect to the head of the screw.

The features of the present invention will be apparent with reference to the following description and attached drawings. In the description and drawings, particular embodiments of the invention have been disclosed in detail as being indicative of some of the ways in which the principles of the invention may be employed, but it is understood that the invention is not limited correspondingly in scope. Rather, the invention includes all changes, modifications and equivalents coming within the spirit and terms of the claims appended hereto.

Features that are described and/or illustrated with respect to one embodiment may be used in the same way or in a similar way in one or more other embodiments and/or in combination with or instead of the features of the other embodiments.

### BRIEF DESCRIPTION OF THE SEVERAL VIEWS OF THE DRAWINGS

FIG. 1A is a cross-sectional view of a locking mechanism of the present invention with a screw and a rod and an intermediate element at or above a temporary stopping point; and

FIG. 1B is a cross-sectional view of the locking mechanism of FIG. 1A in a locked position;

FIG. 1C is another cross-sectional view of the locking mechanism of FIG. 1B in a locked position;

FIG. 2A is a cross-sectional view of part of another locking mechanism of the present invention at a first temporary stopping point;

FIG. 2B is a cross-sectional view of the locking mechanism of FIG. 2A at a second temporary stopping point;

FIG. 2C is a cross-sectional view of the locking mechanism of FIG. 2A in a locked position;

FIG. 3A is a top perspective view of the intermediate element;

FIG. 3B is a bottom perspective view of the intermediate element;

FIG. 3C is a bottom profile view of the intermediate element; and

FIG. 3D is a bottom view of the intermediate element engaged with a screw head.

### DETAILED DESCRIPTION OF THE INVENTION

The invention relates to a novel locking mechanism and method for locking the relative positions of a rod and a screw. The locking mechanism provides an improved lock between the rod and the screw head. The locking element includes a body and intermediate element. The intermediate element is located between the rod and the head of a screw and may directly contact both the rod and screw when the locking mechanism is in a final locking position. The intermediate element preferably has a non-circular shape such that at least one non-contact area exists when the intermediate element is in contact with the head of the screw and the locking mechanism is fully engaged. To engage the locking mechanism, the rod may be preferably urged downward toward the bottom of the body, thereby forcing the intermediate element toward the bottom of the body. The body may also have at least one structure defining one or more distinct temporary stopping points for the intermediate element above the final locking position to facilitate proper alignment of the locking mechanism during use.

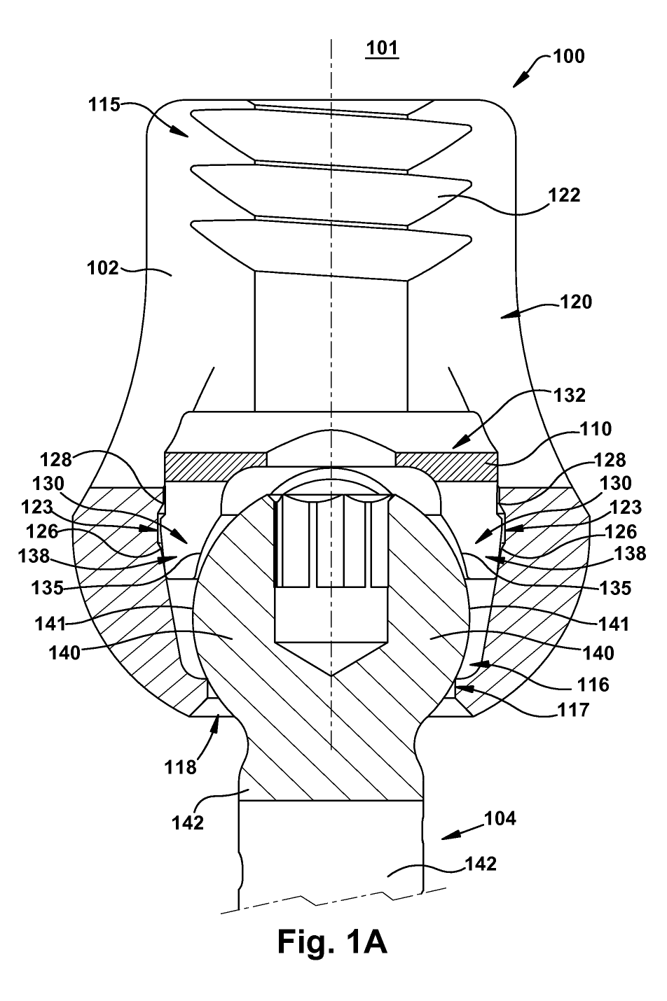

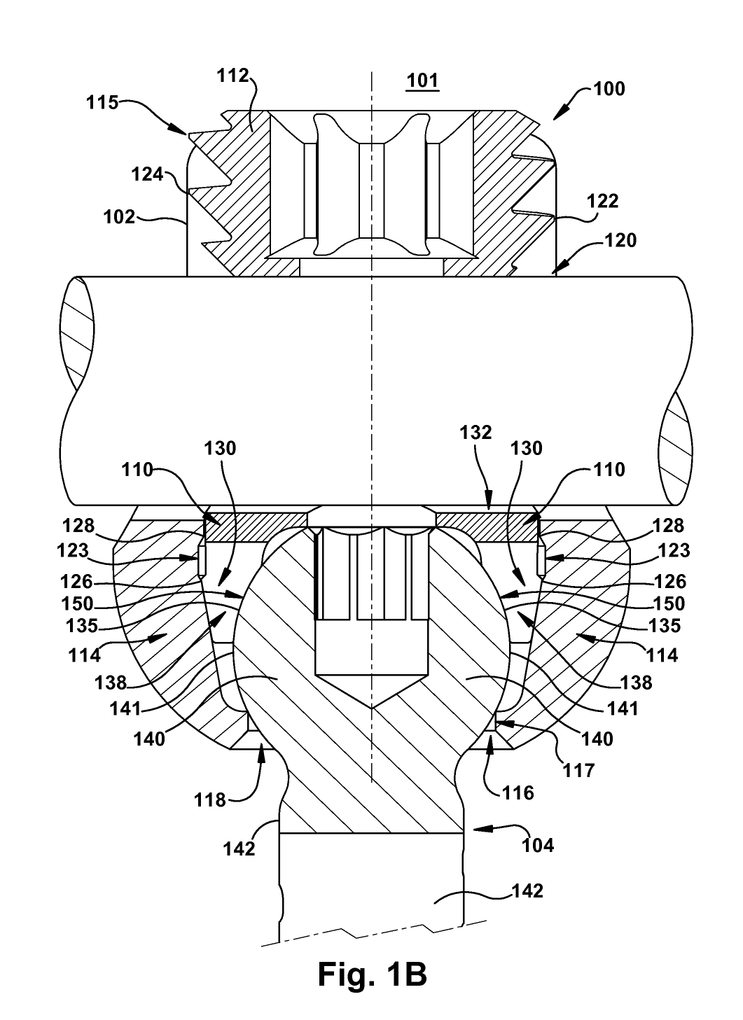

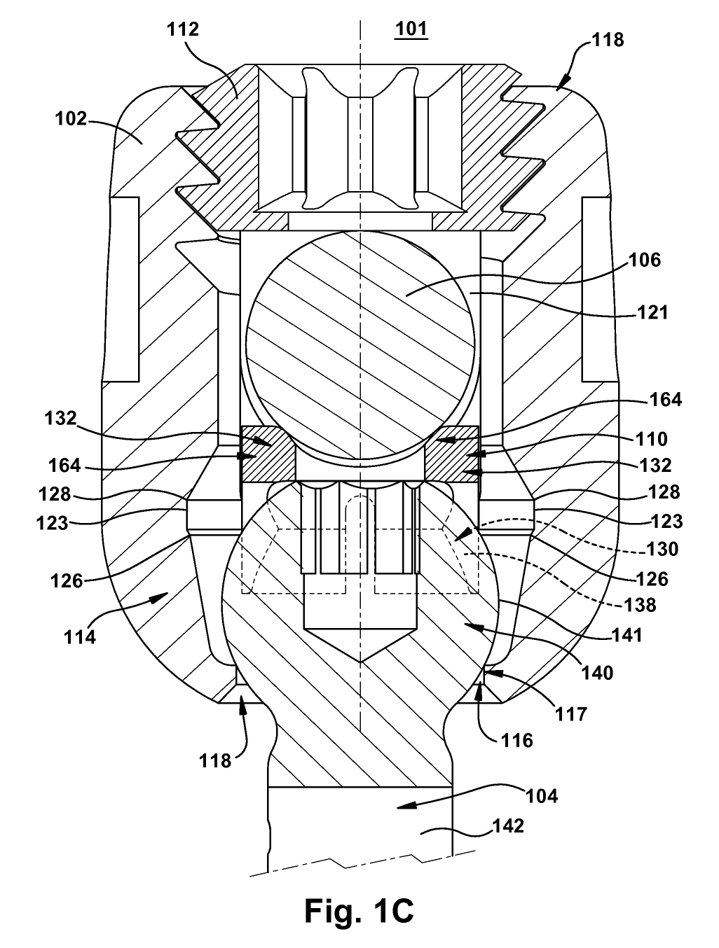

Turning initially to FIGS. 1A-C, a locking mechanism 100 is configured to engage and lock the position of a screw 104 with respect to the position of a rod 106. FIG. 1A is a cross-sectional view of the locking mechanism 100 of the present invention in an unlocked position. FIGS. 1B-C are cross-sectional views of the locking mechanism 100 of the present invention in a locked position, where FIG. 1C is 90 off axis FIG. 1B.

When the locking mechanism 100 is used for spinal fixation, “above” or “top” means posterior with respect to the patient and “below” or “bottom” means anterior with respect to the patient. Thus, the bottom region 114 of the body 102 is anterior with respect to the patient and the rod 106 is received by the body 102 as the rod 106 is moved in a posterior to anterior direction. In addition, because the general shape of the type of body 102 illustrated in FIGS. 1A-C somewhat resembles a tulip flower, a body 102 of the type illustrated is often referred to as a “tulip” by those skilled in the art.

The locking mechanism 100 includes a body 102, intermediate element 110 and locking element 112. The body 102 includes a bottom region 114 having a hole 118 and a socket 116. The socket 116 includes at least one surface 117 surrounding the hole 118. The hole 118 and socket 116 are configured to receive the screw 104 such that the socket 116 engages part of the head 140 of the screw 104 and prevents the screw 104 from passing completely through the hole 118. In other words, the shaft 142 of the screw 104 is placed through the hole 118 and the screw 104 is urged downward until the head 140 of the screw 104 contacts at least one surface 117 surrounding the hole 118, which is large enough to permit passage of the shaft 142 and small enough to prevent passage of the head 140. In addition, the at least one surface 117 may be contoured to match the contour of the surface 141 of the head 140 of the screw 104. Thus, a screw 104 having a shaft 142 and a generally spherical head 140 may be placed through hole in a top region 115 of the body 102 and placed partially through a hole 118 in the bottom region 114 of the body 102 that is smaller in diameter than the head 140 such that the head 140 is caused to contact the at least one surface 117 of the socket 116.

The intermediate element 110 may also be placed through a hole in the top region 115 of the body and urged downward such that the bottom portion 130 of the intermediate element 110 contacts the head 140 of the screw 104. When the intermediate element 110 and screw 104 are forced toward the bottom region 114 of the body 102, the at least one surface 117 of the socket 116 of the bottom region 114 of the body 102 engages the head 140 and prevents the screw 104 from exiting the body 102 through the hole 118, thereby causing increased forces to be exerted on the head 140 by the bottom portion 130 of the intermediate element 110.

The intermediate element 110 may be a “rod seat” having a contoured surface for receiving a rod or a “washer” having a flat surface. Although one of ordinary skill in the art may understand “rod seat” and “washer” to be different types of structures, either may be utilized.

The body 102 also includes a side 120 that is configured to receive the rod 106, such as by way of a channel 121 that enables placement of the rod 106 by either sliding the rod 106 through the side 120 of the body or by inserting the rod 106 into the channel 121 through the top region 115 of the body 102. The intermediate element 110 is preferably inserted into the body 102 prior to insertion of the rod 106 such that the intermediate element 110 is eventually positioned between the rod 106 and the screw 104. In addition, the body 102, screw 104 and intermediate element 110 may be preassembled.

The top portion 132 of the intermediate element 110 may have a tapered portion 164 for receiving the rod 106. Preferably, the center of the tapered portion 164 is aligned with the central axis 101 of the body 102 to facilitate alignment of the rod 106 within the body 102. The tapered portion 164 may also be configured to engage rods of varying diameters, such as rods having diameters ranging from 3 mm to 7 mm. For example, the tapered portion 164 may have multiple curvatures on each side of the taper that provide varying surfaces for contacting rods of varying diameters.

The locking mechanism 100 may also include a locking element 112 that is configured to engage the body 102 and the rod 106 so as to force the rod 106 toward the screw 104. Preferably, the locking element 112 is a set screw, which may be either internally or externally threaded to engage either an externally or internally threaded body 102.

Each of the body 102, the intermediate element 110, the locking element 112, the screw 104 and the rod 106 may be made from a variety of materials known in the art and preferably is made from a biocompatible material when the locking mechanism 100 is used for bone fixation. Such materials include, but are not limited to, titanium, titanium alloys (e.g. titanium/aluminum/vanadium (Ti/Al/V) alloys), cobalt-chromium alloys, stainless steel, ceramics (alumina ceramic, zirconia ceramic, yttria zirconia ceramic, etc.), high strength polymers (e.g. PEEK, PEKK, etc.), pyrolytic carbon, tantalum, carbon composite materials and combinations thereof, which may include mechanically compatible mixtures of the above materials. Such materials are commonly used in bone fixation and the like. Preferably, the materials are rigid and in one embodiment, the body 102, screw 104, rod 106, intermediate element 110 and locking element 112 are all made from Ti/Al/V alloys, such as Ti/6Al/4V ELI.

The size of the body 102 may be similar to that of known devices. For example, the height of body 102 may range from about 0.4 inch to about 1 inch. Also, the width of body 102 may range from about 0.25 inch to about 1 inch. The body 102 also may include a pocket 123 for engaging the intermediate element 110 and maintaining proper alignment of the intermediate element 110 within the body 102. The pocket 123 may be, for example, wider than the body 102 immediately below the pocket 123. Likewise, the intermediate element 110 may be configured to engage the pocket 123 when placed in the body 102. In one embodiment, the intermediate element 110 is snappably engageable with the pocket 123, such as by snapping into a recessed groove in the pocket 123. In addition, the body 102 is preferably configured to accept an intermediate element 110 having a non-circular shape and maintain the orientation of the intermediate element 110 with respect to the body 102.

For example, the body 102 may have at least one structure, such as a pocket 123, groove, ridge, sloped surface or the like that defines at least one distinct temporary stopping point for the intermediate element above the final locking position of the intermediate element 110 shown in FIG. 1B. The at least one structure may, for example, impede the progress of the intermediate element 110 toward the bottom of the body 102, thereby providing feedback to a user that the intermediate element 110 has reached a distinct temporary stopping point. As illustrated in FIG. 1A, the body 102 includes a pocket 123 having a ridge 126 that creates a distinct temporary stopping point for the intermediate element 110. The position of the intermediate element 110 at the distinct temporary stopping point defined by the pocket 123 and ridge 126 may be, for example, about 2 mm to about 6 mm from the final locking position of the intermediate element 110 at the final locking position shown in FIG. 1B.

In addition, the pocket 123 may be configured to prevent the intermediate element 110 from rotating about the central axis 101. The body 102 may also include a structure, such as ridge 128 for preventing the intermediate element from migrating toward the top of the body 102.

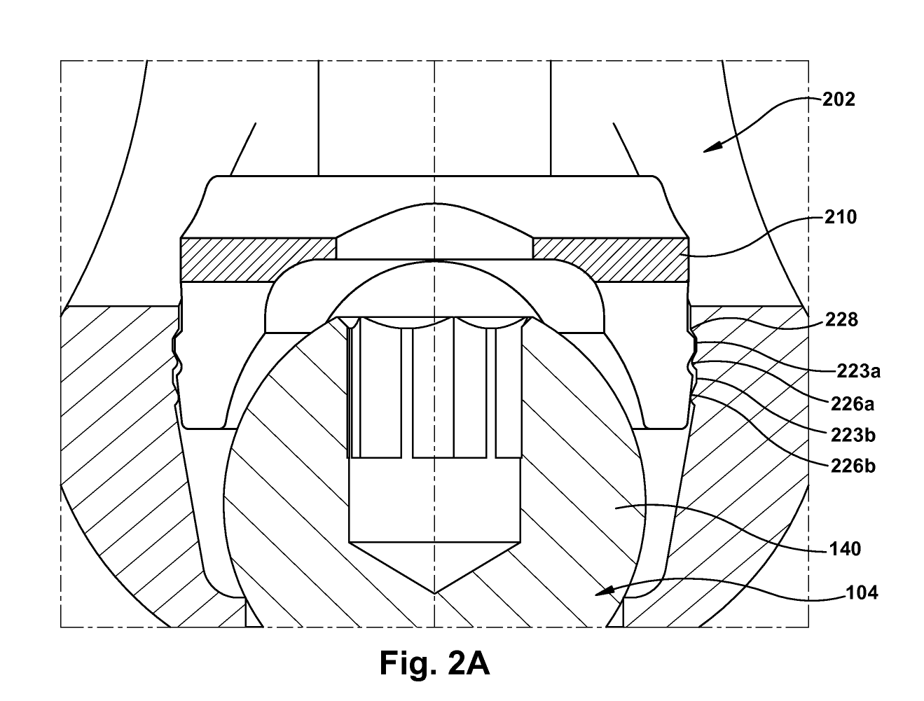

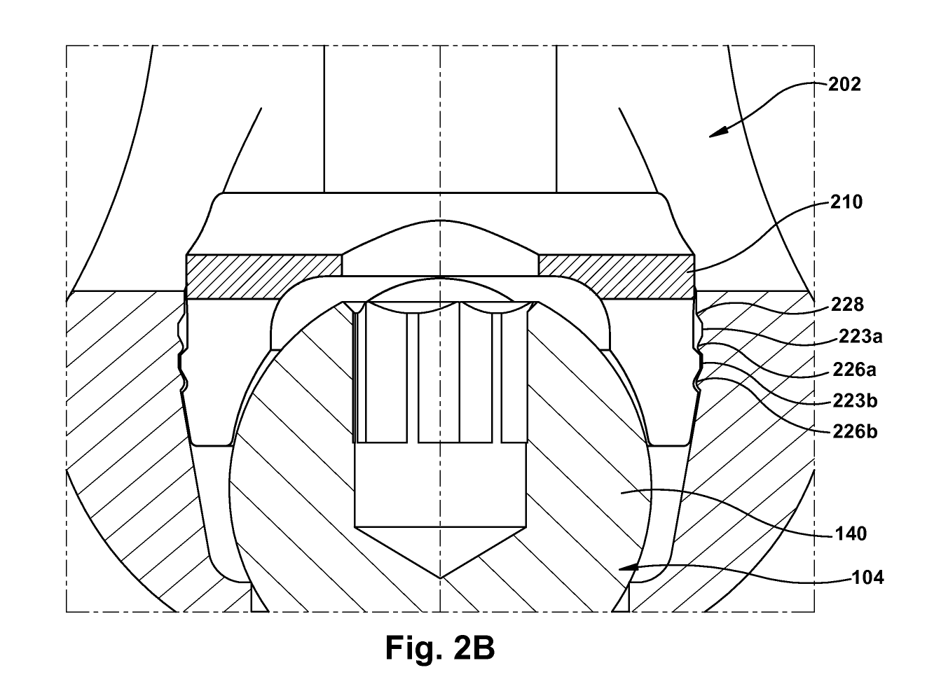

The intermediate element 110 is shown in greater detail in FIGS. 2A-D. The intermediate element 110 may serve multiple functions, such as aiding in the alignment of the rod 106, creating a contact surface for the rod 106, exerting forces on the head 140 that have both vertical and lateral components, and preventing linear compressive forces from being transferred from the rod 106 to the top of the head 140.

To aid in the alignment of the rod 106 and provide a contact surface for the rod 106, the intermediate element 110 may have a taper 164. Preferably, the midline of the taper 164 is aligned with the central axis 101 of the body 102. In this manner, the taper 164 facilitates placement and alignment of the rod 106 within the body 102 such that the center of the rod 106 is generally aligned with the central axis 101 of the body 102. It should be understood by those of ordinary skill in the art that the intermediate element 110 may also have a non-tapered surface for contacting the rod 106.

In addition, the taper 164 may include multiple curvatures, such as curvatures 166 and 168 on each side of the intermediate element 110. The multiple curvatures may create engagement surfaces for rods of varying diameter. In the preferred embodiment, the intermediate element 110 is configured to engage a rod 106 where the rod 106 has a diameter ranging between 3 mm and 7 mm. The diameter of the rod 106 may determine which of the curvatures contacts the rod 106. For example, the surface of a first curvature may be the primary engagement surface for a 3 mm rod 106, but the surface of a second curvature may be the primary engagement surface for a 7 mm rod 106. In addition, it will be understood by those skilled in the art that the surfaces of curvatures may overlap and that a rod 106 may contact the surface of both curvatures. The taper 164 may also accommodate rods of varying diameter without having multiple curvatures by providing an extended contact surface having a single curvature where rods of larger diameters contact the extended surface closer to the top of the taper 164 than do rods of smaller diameters.

The intermediate element 110 preferably has a non-circular shape. For example, the intermediate element 110 may be ovular as illustrated in FIGS. 2A-D, or have only a bottom profile that is ovular or rectangular. In addition, the profile of the intermediate element 110 may vary from top to bottom of the intermediate element 110. For example, the intermediate element 110 may have a generally non-circular bottom profile (e.g., ovular or rectangular) while having a top profile that is a different shape (e.g., generally circular, or generally hexagonal). Also, the bottom portion 130 of the intermediate element 110 may have a length that is greater than the width. In one embodiment, the bottom portion 130 of the intermediate element 110 has a length that is greater than the width, wherein the length is generally parallel to the major axis of the rod 106.

In one embodiment, intermediate element 110 includes at least two feet 138, and may include at least two pairs of feet 138. Between the two feet 138 or two sets of feet 138 is a non-contact area 136 that preferably spans at least about 40 degrees of the circumference of the head 140. The feet 138 may be generally opposite each other with respect to a major axis of the intermediate element 110, such as illustrated in FIGS. 2A-D. The feet 138 also may be generally offset from one another. Each of the feet 138 preferably has a generally curved contact surface 135 that defines a contact area 150 when the contact surface 135 of the foot 138 is brought into contact with the surface 141 of the head 140. At least part of the contact surface 135 may be adapted to match the contour of the surface 141 of the head 140 to increase the contact area 150. In addition, the feet 138 may be configured such that the length L of the contact area 150 in the vertical dimension is at least 20 percent the width W of the contact area 150 in the horizontal dimension.

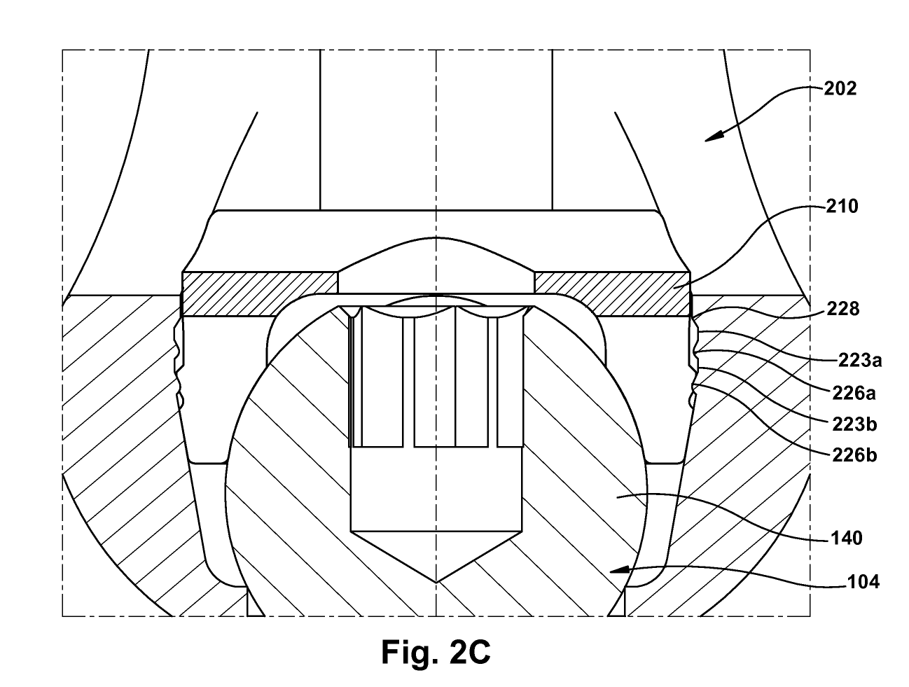

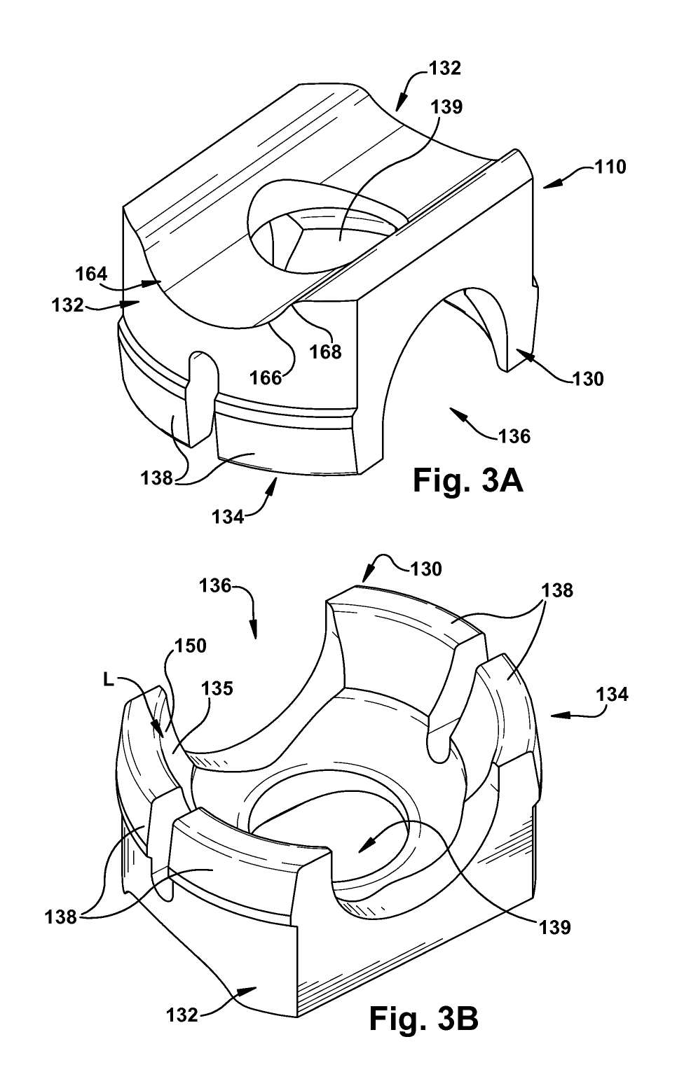

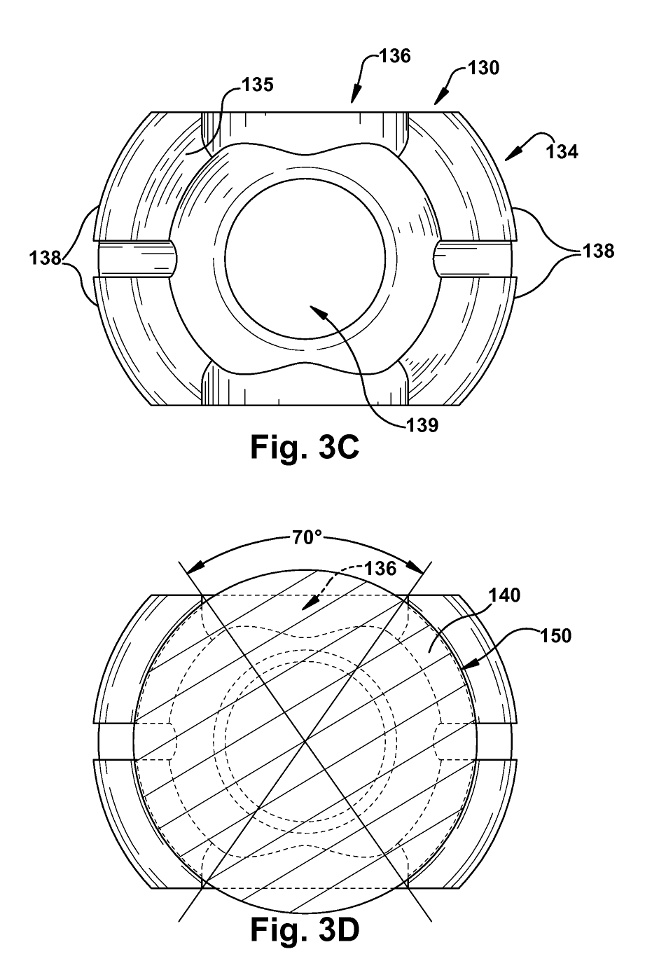

In addition, as shown in FIGS. 3A-D, the non-circular shape and position of the intermediate element 110 above the screw 104, as well as the placement of the feet 138 such that they are equidistant from the major axis of the rod 106 and generally opposite one another with respect to the major axis of the rod 106 may counter twisting/rotational forces placed on the rod 106 following locking.

As shown in FIGS. 3A-D, the intermediate element 110 has two non-contact areas 136, each of which has a width of more than about 40 degrees of the circumference of the head 140. Other embodiments may include at least one non-contact area 136 having a width ranging from about 55 degrees to about 95 degrees of the circumference of the head 140. A second of the at least one non-contact areas 136 may also have a width ranging from about 55 degrees to about 95 degrees of the circumference of the head 140.

The intermediate element 110 may also have a bottom portion 130 including a generally circular part 134, and a generally non-circular part (which is represented in FIGS. 3A-D as the non-contact area 136). The generally circular part 134 may be configured to contact the head 140 and the generally non-circular part 136 may be configured so as to not contact the head 140.

Due to its generally non-circular bottom profile, the intermediate element 110 may have less “hoop strength” than a similar circular element. Thus, the intermediate element 110 having a generally non-circular bottom profile may provide less resistance to compressive forces and increased transfer of forces to the head 140 of the screw 104. Moreover, the feet 138 may be relatively small compared to the circumference of the head of the screw, which may provide less resistance to deformation of the feet 138. Thus, given the same downward force on the top of the intermediate element 110, a non-circular bottom profile feet 138 may provide greater deformation than a circular bottom, profile thereby resulting in a greater friction between the contact surfaces 135 of the feet 138 and the surface 141 of the head 140 of the screw 104.

The intermediate element 110 also may include a hole 139 to provide access to the head 140 of the screw 104 from the top of the body 102. Thus, a driver or similar device may be used to engage the head 140 of the screw 104 via the intermediate element 110.

In use, the body 102, the screw 104 and the intermediate element 110 may be preassembled. Alternatively, the screw 104 may be inserted first and the intermediate element 110 inserted second, preferably through the top of the body 102. Preferably, the intermediate element 110 is not in contact with the screw 104 and may be, for example, about 2 mm to about 6 mm from the screw 104. The intermediate element 110 may be, for example, in a pocket or at a temporary stopping point that prevents the unintended movement of the intermediate element 110 toward the bottom of the body 102. At this point of the process the screw 104 is capable of polyaxial movement with respect to the body 102.

A driver may then be inserted into the body 102 such that the driver engages the head 140 of the screw 104 via the hole 139 in the intermediate element 110. The screw 104 then may be driven into a desired location, such as into a desired location of the spine. At this point the screw 104 is fixed with respect to the desired location, but the screw 104 is capable of polyaxial movement with respect to the body 102. Thus, the position of the body 102 may be adjusted with respect to the position of the screw 104.

Following tightening of the screw 104, the rod 106 may be received by the body 102. The body 102 may include a channel in the side for receiving the rod 106, which may be a dynamic stabilization rod. A locking element 112, which is preferably a set screw, may then be used to initiate locking. Although internal threads 122 are illustrated in FIGS. 1A-C, the treads may be either internal or external to the body 102 depending on the configuration of the body 102 and the locking element 112. For example, it will be understood by those of ordinary skill in the art that the threads may also be external threads and the locking element 112 may surround the body 102 during engagement.

The locking element 112 may be thus engaged with the threads 122 to keep the rod 106 within the channel 121. It should be understood, however, that the locking element 112 need not engage the body 102 via threaded engagement. The locking element 112 and the body 102 may be slidably engageable, rotatably engageable, and/or snapably engageable. In the embodiment disclosed in FIGS. 1A-C the locking element 112 and the body 102 are rotatably engageable. To fix the rod 106 with respect to the screw 104, the locking element 112 may be tightened down to apply increasing force to the rod 106 in order to engage and lock the rod 106 and screw 104. More specifically, the tightening of the locking element 112 causes linear compression of the rod 106 onto the intermediate element 110, which in turn causes the intermediate element 110 to engage to the head 140 of the screw 104 and forces the screw 104 toward the bottom of the body 102.

Although the rod 106 forces the intermediate element 110 downward, the manner in which the intermediate element 110 engages the head 140 may prevent the transfer of linear compressive forces from the intermediate element 110 to the head 140. For example, the forces exerted by the bottom portion 130 of the intermediate element 110 on the head 140 may be off axis of the central axis 101 of the body 102. In one embodiment, the force exerted on the head 140 ranges from about 10 degrees to about 80 degrees off the central axis 101 of the body 102. In one embodiment, the force exerted on the head 140 is about 50 degrees off the central axis 101 of the body 102.

More specifically, the locking element 112 may urge the rod 106 toward the bottom of the body with sufficient force to cause the intermediate element 110 to pass below the temporary stopping point. The force required to cause the intermediate element 110 to pass below the temporary stopping point may act as a feedback mechanism for the user. As illustrated in FIGS. 1A-C, force applied to the intermediate element 110 causes the intermediate element to exit the pocket 123 and enter into a tapered area of the body 102 where friction between the intermediate element 110 and the body 102 increases as the intermediate element 110 is urged toward the bottom of the body 102. The tapered shape of the body 102 transfers the downward linear force, resulting from the tightening of the locking element 112, to an off axis locking force that engages the head 140 of the screw 140. After exiting the pocket 123, the screw 104 may be monoaxial with respect to the body, but still reversible. Eventually, as the intermediate element 112 is urged downward, the monaxial nature of the screw 104 and is no longer reversible. At this point, the intermediate element 110 is in a final locking position and the position of the rod 106 is locked with respect to the position of the screw 104.

Thus, the head 140 is urged toward the socket 116, which is also configured for engagement with the head 140. In order to facilitate locking engagement, the surface of the socket 116 may include rough or knurled surface and/or a surface fixation mechanism, such as ridges, grooves, bumps, pips, or the like to increase the coefficient of friction of the surface. In addition, the bottom portion of the intermediate element 110 as well as the head 140 may have rough or knurled surfaces and/or surface fixation mechanisms, such as ridges, grooves, bumps, pips, or the like to increase the coefficient of friction of the surfaces. Preferably, the head 140 is textured. For example, the surfaces may roughened by blasting, for example, with titanium oxide, glass beads or other suitable blasting material. One of skill in the art will understand that other surface treatments may also be used on the surfaces of the socket 116, the insert 108 and the head 140. In one embodiment, the head 140 is textured but has no grooves.

Thus, as the rod 106 is forced downward, the intermediate element 110 exerts forces on the upper hemisphere of the head 140 that have both lateral and vertical components and the at least one surface 117 exerts forces on the lower hemisphere of the head 140 that have both lateral and vertical components. In addition, because of the non-contact area 136 in the bottom portion 130 of the intermediate element 110, the forces are only applied to the head 140 by the intermediate element 110 at each of the feet 138 at the respective contact area 150. The respective contact areas 150 also may be generally opposed to create a squeezing effect from generally opposing locations on the head 140.

Turning next to FIGS. 2A-C, another embodiment of an intermediate element 210 and body 202 are illustrated. The intermediate element 210 and body 202 are the same in all aspects as the intermediate element 110 and body 102 except that the intermediate element 210 is adapted for use with a body 202 having multiple structures defining multiple distinct temporary stopping points.

The body 202 also may include multiple pockets 223 for engaging the intermediate element 110 and maintaining proper alignment of the intermediate element 210 within the body 202. The pockets 223 may be, for example, wider than the body 202 immediately below the pockets 223. Likewise, the intermediate element 210 may be configured to engage the pockets 223 when placed in the body 202. The intermediate element 210 may be snappably engageable with the pockets 223, such as by snapping into recessed grooves in the pockets 223. In addition, the body 202 is preferably configured to accept an intermediate element 210 having a non-circular shape and maintain the orientation of the intermediate element 210 with respect to the body 202. The body 202 may also include a structure, such as ridge 228 for preventing the intermediate element 210 from migrating toward the top of the body 202.

As shown, the body 202 has multiple structures, such as pockets 223a and 223b, having ridges 226a and 226b, respectively, defining first and second distinct temporary stopping points for the intermediate element 210. As shown in FIG. 2A, the position of the intermediate element 210 at the first distinct temporary stopping point defined by the pocket 223a and ridge 226a may be, for example, the preassembled position of the intermediate element 210 and body 202. The first temporary stopping point may be about 2 mm to about 6 mm from the final locking position. The screw 104 is polyaxial with respect to the body and may be driven into a desired location when the intermediate element 210 is at the first temporary stopping point. Following the fixation of the screw 104, the intermediate element 210 may be further urged toward the bottom of the body 202, causing the intermediate element 210 to pass below the first temporary stopping point. The force required to cause the intermediate element 210 to pass below the temporary stopping point may act as a feedback mechanism for the user. As shown in FIG. 2B, the intermediate element 210 may thus be urged to a second temporary stopping point. At the second temporary stopping point the bottom portion of the intermediate element 210 is caused to contact the head 140 of the screw 104, but the screw 104 is still polyaxial with respect to the body 202 and the body 202 may be repositioned with respect to the screw 104.

The intermediate element 210 may be further urged beyond the second temporary stopping point such that the bottom portion of the intermediate element enters a tapered portion of the body 202. As the intermediate element 210 is advanced toward the head 140 of the screw 104, the taper lock between the intermediate element 210 and the body 202 preferably ensures that the intermediate element 210 maintains its position and maintains friction of the head 140 of the screw 104. This position may not be a fixed snap-in position. Rather, the amount of friction may depend on how far the intermediate element 210 is advanced and may be tailored to the needs of the user. Prior to reaching the final locking position, the body 202 may be moveable and repositionable with respect to the screw 104, but capable of holding its poly-axial orientation.

Finally, the intermediate element 210 may be urged to its final locking position as illustrated in FIG. 6C, in which the forces applied to the head 140 of the screw 104 are similar to those applied by the intermediate element 110 and body 102 as illustrated in FIG. 1C.

While the present invention has been described in association with exemplary embodiments, the described embodiments are to be considered in all respects as illustrative and not restrictive. Such other features, aspects, variations, modifications, and substitution of equivalents may be made without departing from the spirit and scope of this invention which is intended to be limited only by the scope of the following claims. Also, it will be appreciated that features and parts illustrated in one embodiment may be used, or may be applicable, in the same or in a similar way in other embodiments.

Although the invention has been shown and described with respect to certain embodiments, it is obvious that certain equivalents and modifications may be apparent to those skilled in the art upon the reading and understanding of the specification. The present invention includes all such equivalents and modifications, and is limited only by the scope of the following claims.

What is claimed is:

1. An intermediate element for use with a locking mechanism comprising:
a top portion adapted to receive a rod; and
a bottom portion adapted to receive a generally spherical head of a screw, the bottom portion comprising:
at least two feet, each of the feet having a generally curved contact surface at least part of which is adapted to match the contour of a surface of the head of the screw such that a contact area is defined when the foot is brought into contact with the surface of the head, and
at least one non-contact area between two of the at least two feet spanning at least about 40 degrees of the circumference of the head of the screw.

2. The intermediate element of claim 1 wherein the intermediate element has a generally non-circular bottom profile.

3. The intermediate element of claim 2 wherein the intermediate element has a generally ovular bottom profile.

4. The intermediate element of claim 3 wherein part of the intermediate element is generally circular and part of the intermediate element is generally non-circular, wherein the generally circular part of the intermediate element contacts the head of the screw, and wherein the generally non-circular part of the intermediate element does not contact the head of the screw.

5. The intermediate element of claim 1 comprising at least two pairs of feet and at least two non-contact areas between the two pairs of feet, wherein each of the two non-contact areas has a width of at least about 40 degrees of the circumference of the head of the screw.

6. The intermediate element of claim 1 wherein the at least one non-contact area has a width of from about 55 degrees to about 95 degrees of the circumference of the head of the screw.

7. The intermediate element of claim 1 wherein each of contact areas has a vertical and a horizontal dimension and wherein a length of the contact area in the vertical dimension is at least 20 percent of a width of the contact area in the horizontal direction.

8. The intermediate element of claim 1 wherein the sum of the widths of the contact areas on the head of the screw is less than about 280 degrees of the circumference of the head of the screw.

9. The intermediate element of claim 1 wherein at least one of the feet is located at a position generally opposite a position of another of the feet of the intermediate element.

10. The intermediate element of claim 1 wherein the feet are offset from one another such that none of the feet is centered at a position opposite a position of a center of another of the feet of the intermediate element.

11. The intermediate element of claim 1 wherein the top portion of the intermediate element is adapted to receive a rod selected from a group of rods of varying diameters.

12. A locking mechanism comprising:
a body having a bottom region and a side, the side portion being configured to receive a rod;
a screw extending at least partially through a hole in the bottom region of the body; and
an intermediate element between the rod and the screw, the intermediate element having a top portion adapted to receive a rod and a bottom portion adapted to receive a head of a screw;
wherein the body comprises a pocket adapted to receive the intermediate element and maintain the orientation of the intermediate element with respect to the body.

13. The locking mechanism of claim 12 wherein the intermediate element has a non-circular bottom profile.

14. The locking mechanism of claim 13 wherein the bottom profile of the intermediate element comprises a width and a length, wherein the length is greater than the width and wherein the length is generally parallel to a major axis of the rod.

15. The locking mechanism of claim 12 wherein the bottom portion of the intermediate element comprises at least two contoured surfaces, and wherein at least part of each of the contoured surfaces is adapted to match the contour of the surface of the head of the screw to define a contact area on the surface of the head when in contact with the head.

16. The locking mechanism of claim 15 wherein the bottom portion of the intermediate element further comprises a non-contact area between two of the at least two contoured surfaces, wherein the non-contact area has a width of at least about 40 degrees of the circumference of the head of the screw.

17. The locking mechanism of claim 15 wherein the bottom portion of the intermediate element comprises two non-contact areas between the at least two contoured surfaces, wherein each of the two non-contact areas has a width of from about 50 degrees to about 100 degrees of the circumference of the head.

18. The locking mechanism of claim 15 wherein at least two of the contoured surfaces are opposite one another with respect to a major axis of the intermediate element.

19. A locking mechanism comprising:
a body having a bottom region and a side adapted to receive a rod;
a screw extending at least partially through a hole in the bottom region of the body; and
an intermediate element adapted for placement between the rod and the screw;
wherein locking is accomplished by urging the rod toward the bottom of the body until the intermediate element reaches a final locking position; and
wherein the body comprises at least one structure defining at least one distinct temporary stopping point for the intermediate element above the final locking position.

20. The locking mechanism of claim 20 wherein the body comprises two distinct temporary stopping points for the intermediate element above the final locking position.

21. The locking mechanism of claim 20 wherein the at least one structure impedes the progress of the intermediate element toward the bottom of the body, thereby providing feedback to a user that the intermediate element has reached a distinct temporary stopping point.

22. The locking mechanism of claim 20 wherein the at least one structure comprises a pocket within the body that is wider than the body immediately below the pocket.

23. The locking mechanism of claim 20 wherein the intermediate element is in direct contact with the screw and the rod when the intermediate element reaches the final locking position.

24. The locking mechanism of claim 20 wherein the intermediate element is not in contact with the screw when the intermediate element is located at least one stopping point.

25. A method for locking the relative positions of a rod and a screw comprising:
inserting a driver into a body at least partially encompassing a screw and an intermediate element located at or above a distinct temporary stopping point defined by at least one structure in the body above the screw;
engaging the driver and the screw via a hole in the intermediate element;
driving the screw into a desired location;
adjusting the position of the body with respect to the position of the screw;
placing a rod in the body and urging the rod toward bottom of the body with sufficient force to cause the intermediate element to pass below the temporary stopping point; and
further urging the rod toward the bottom of the body to cause the intermediate element to exert force on the head of the screw, thereby locking the position of the rod with respect to the position of the screw.

26. The method of claim 25 wherein the intermediate element is not in contact with the screw when the screw is engaged by the driver.

27. The method of claim 25 wherein the intermediate element is located in a pocket of the body when the screw is engaged by the driver.

28. The method of claim 27 wherein the intermediate element has a non-circular bottom profile and the pocket of the body is adapted to receive the intermediate element and prevent rotation of the intermediate element about a major axis of the body.

29. The method of claim 25, further comprising urging the intermediate element to a second distinct temporary stopping point defined by at least one structure in the body, the second distinct stopping point being below the first distinct stopping point and above the screw.

30. The method of claim 25 wherein the intermediate element is caused to exert force on the head of the screw over at least two contact areas separated by a non-contact area having a width of from about 50 degrees to about 100 degrees of the circumference of the head of the screw.

31. The method of claim 25 wherein the intermediate element is caused to exert force on the head of the screw over at least two contact areas that are generally located opposite one another with respect to the head of the screw.

32. A method for locking the relative positions of a rod and a screw comprising:
placing a rod in a body at least partially encompassing a screw an intermediate element located above the screw, wherein the intermediate element has a non-circular bottom profile and the body is adapted to receive the intermediate element and prevent rotation of the intermediate element with respect to the body about a major axis of the body; and
urging the rod toward the bottom of the body and causing the intermediate element to exert force on the head of the screw, thereby locking the position of the rod with respect to the position of the screw.

33. The method of claim 32 wherein the intermediate element is caused to exert force on the head of the screw over at least two contact areas separated by a non-contact area having a width of from about 50 degrees to about 100 degrees of the circumference of the head of the screw.

34. The method of claim 32 wherein the intermediate element is caused to exert force on the head of the screw over at least two contact areas that are generally located opposite one another with respect to the head of the screw.
